(19) United States
(12) Patent Application Publication
(10) Pub. No.: US 2025/0134823 A1
(43) Pub. Date: May 1, 2025

(54) ANTI-COAGULATING HYDROGEL NANOFILM FOR CELL ENCAPSULATION

(71) Applicant: Seoul National University R&DB Foundation, Seoul (KR)

(72) Inventors: Nathaniel Suk-Yeon HWANG, Seoul (KR); Young-sun LEE, Seoul (KR); Kyoung-Ha SO, Seoul (KR); Dong-Hyun LEE, Seoul (KR); Seh Ri OH, Seoul (KR); Suwan JEON, Seoul (KR)

(21) Appl. No.: 18/931,785

(22) Filed: Oct. 30, 2024

(30) Foreign Application Priority Data
Oct. 31, 2023 (KR) 10-2023-0147573
Feb. 19, 2024 (KR) 10-2024-0023527

(51) Int. Cl. A61K 9/51; C12N 5/00

(52) U.S. Cl. CPC A61K 9/5161; A61K 9/5192; C12N 5/0012

(57) ABSTRACT

The present invention relates to a method for modifying and cross-linking an anticoagulating hydrogel nanofilm for cell encapsulation. According to the present invention, the protection from the external environment and the control of material transfer can be improved due to the anticoagulant functionalization of the material surface. In addition, when applied into the body, it can prevent fibrosis, blood coagulation, protein adsorption, and platelet adhesion caused by immune responses, thereby preventing a decrease in cell viability caused by immune responses and improving cell survival rate.

### CROSS REFERENCE TO RELATED APPLICATIONS

This application claims the benefit of priorities to Korean Patent Application Nos. 10-2023-0147573 filed on Oct. 31, 2023, and 10-2024-0023527 filed on Feb. 19, 2024, the entire disclosure of which are incorporated herein by reference.

### RESEARCH FUNDING ACKNOWLEDGEMENT

In addition, this application is based on the research results conducted by the research project of Korean Fund for Regenerative Medicine “Development of hydrogel nanofilm encapsulation technology to regulate in vivo immune response for xenogeneic pancreatic beta cell transplantation” (Serial No.: 1711177404, Detailed Serial No.: 21A0301L1-12).

### TECHNICAL FIELD

The present invention relates to a method for modifying and cross-linking an anticoagulating hydrogel nanofilm for cell encapsulation.

### BACKGROUND ART

Cell therapeutic agents are drugs that transplant or inject cells such as a stem cell, an immune cell, and a beta cell into the body, and are attracting attention for treatment of severe diseases. Commercial interest in the cell therapeutic agents that use the cells, as pharmaceuticals, such as a CAR-T cell, which is the representative cell therapeutic agent for a blood cancer, the beta cell, which is the cell therapeutic agent for type 1 diabetes, and the stem cell therapeutic agent, is growing to apply these cells to a liver cirrhosis, an arthritis, and a cancer that are difficult to treat in the modern medical field.

Hormonal or drug therapy is a temporary treatment for various endocrine diseases. To overcome the shortcomings of these temporary treatments, research is ongoing to restore homeostasis through the transplantation of endocrine cells into the body as a semi-permanent treatment. Since the cells used as the therapeutic agents have a problem in that a survival rate thereof is greatly reduced due to physical stress and immune response when transplanted into the body, a technology for cell encapsulation has been studied in the fields of a tissue engineering and a regenerative medicine. Conventional platforms for cell delivery include a microbead technology using an alginic acid, a layered coating technology for the cells, and the like.

However, when producing capsules with a thickness of 0.5 to 1.5 mm using alginate to use treat type 1 diabetes, clinical results are limited due to problems such as fibrosis on the capsule surface and lack of ability to suppress inflammatory cytokine when applying long-term blood glucose treatment. The microbead technology has a problem in that supply and exchange of materials outside the capsule, such as a nutrient and oxygen, are limited due to a hydrogel too thick compared to a size of the cells, so that the cells are necrotic or their function is deteriorated. In addition, since the layered coating technology for the cells utilizes an attraction force between negatively and positively charged polymers, which results in generating a relatively weak electrostatic force in the bonding strength, the thickness of the coated layers is relatively thin so that the coated layers can be easily removed by a physical stress from the outside such as a pressure during the injection process and a blood flow, whereby there is limitation in stability, persistence, and durability. Using such bulky capsules needs to find a new transplant site because it cannot be applied to the Edmonton protocol which is an approved surgical method for infusion of pancreatic islets into the bloodstream through the portal vein.

Therefore, a technology is needed that can effectively control the inflammatory reaction caused by the immune system and the coagulation reaction upon contact with blood when transplanting cells into the body through vascular injection such as the portal vein, thereby improving the survival rate.

### DETAILED DESCRIPTION OF THE INVENTION

### Technical Problem

It is an object of the present invention to impart anticoagulant functionality to the surface by modifying the polymer of the outermost layer of a cell cluster capsule using a multi-layer coating.

### Solution to Problem

In order to solve the above problem, the present invention provides a modified anticoagulating hydrogel for cell encapsulation, comprising:
a chitosan layer comprising a functionalized chitosan;
a hyaluronic acid layer comprising a functionalized hyaluronic acid; and
an outermost layer comprising a functionalized sulfated polymer,
wherein the chitosan layer and the hyaluronic acid layer are alternately layered,
the chitosan layer and the hyaluronic acid layer form aryloxy cross-linking with each other by an enzymatic reaction of tyrosinase, and
the sulfated polymer comprises at least one of chondroitin sulfate, sulfated hyaluronic acid, and heparin.

According to one embodiment, the thickness of the hydrogel may be 100 nm to 200 nm.

In one embodiment, the chitosan, the hyaluronic acid and the sulfated polymer can each be independently functionalized with phenol, catechol or quinone. Specifically, for example, a monophenol group may be introduced into each of the chitosan, hyaluronic acid and sulfated polymer.

According to one embodiment, the hydrogel for cell encapsulation may comprise 1 to 5 chitosan layers and 1 to 5 hyaluronic acid layers.

According to one embodiment, the hydrogel may be for transplantation of isolated β-cells.

According to other aspect of the invention, it is provided a method for producing a modified anticoagulating hydrogel for cell encapsulation, comprising the steps of:
(i) adding a solution containing a functionalized chitosan and a solution containing a functionalized hyaluronic acid to form layers having a chitosan layer and a hyaluronic acid layer alternately layered, wherein the chitosan layer and the hyaluronic acid layer form aryloxy cross-linking with each other by using tyrosinase enzyme; and
(ii) adding a solution containing a functionalized sulfated polymer onto the layers obtained from step (i) to form an outermost layer, wherein the outermost layer forms aryloxy cross-linking by using tyrosinase enzyme,
wherein the sulfated polymer comprises at least one of chondroitin sulfate, sulfated hyaluronic acid, and heparin.

According to one embodiment, the solution containing a functionalized chitosan may have a concentration of 0.01% (w/v) to 5% (w/v), the solution containing a functionalized hyaluronic acid may have a concentration of 0.01% (w/v) to 5% (w/v), and the solution containing a functionalized sulfated polymer may have a concentration of 0.01% (w/v) to 5% (w/v).

According to one embodiment, the method may be for transplantation of isolated cells.

According to one embodiment, the tyrosinase enzyme may include Streptomyces avermitilis-derived tyrosinase.

According to other aspect of the present invention, it is provided a cell therapy composition for isolated cell transplantation, comprising the modified anticoagulating hydrogel for cell encapsulation as described above and isolated cell.

According to another aspect of the present invention, it is provided an isolated cell encapsulated with the modified anticoagulating hydrogel for cell encapsulation as described above.

Specific details of other embodiments of the present invention are included in the detailed description below.

### Effect of the Invention

According to the present invention, the protection from the external environment and the control of material transfer can be improved due to the anticoagulant functionalization of the material surface. In addition, when applied into the body, it can prevent fibrosis, blood coagulation, protein adsorption, and platelet adhesion caused by immune responses, thereby preventing a decrease in cell viability caused by immune responses and improving cell survival rate.

### BRIEF DESCRIPTION OF THE DRAWING

FIG. 1 is a chemical formula showing a process of introducing a phenol moiety into a polymer.

FIGS. 2A and 2B are structural comparison diagrams the conventional mushroom-derived tyrosinase and actinomycete-derived tyrosinase.

FIG. 3 is a schematic diagram illustrating a process of cross-linking a polymer having a phenol moiety introduced thereto.

FIG. 4 shows the results of confirming that a monophenol moiety is introduced into a glycol chitosan and a hyaluronic acid polymer.

FIG. 5 is graphs showing a reaction rate between a polymer and tyrosinase.

FIG. 6 is a schematic diagram illustrating a process of cell encapsulation.

FIG. 7 is a diagram schematically illustrating encapsulation principle.

FIG. 8 is a schematic diagram and a photograph showing encapsulated cells according to the present invention.

FIG. 9 is photographs observing clustering phenomenon between the cells according to GC-T concentration.

FIG. 10 is a graph confirming cytotoxicity of actinomycetes-derived tyrosinase.

FIG. 11 is graphs showing the degree of cell encapsulation depending on concentration of an enzyme, reaction time, and concentration of a polymer.

FIG. 12 is graphs showing whether or not cell encapsulation is formed according to tyrosinase and electrostatic force.

FIG. 13 is a graph confirming whether or not a hydrogel nanofilm is formed by a Quartz Crystal Microbalance.

FIG. 14 is a graph showing zeta potential of encapsulated cells.

FIG. 15 is a graph showing FACS results of encapsulated cells.

FIG. 16 is photographs showing CLSM results of an encapsulated single cell.

FIG. 17 is photographs showing CLSM results of encapsulated β-cell spheroids.

FIG. 18 is photographs showing SEM results of encapsulated cells.

FIG. 19 is photographs showing TEM results of encapsulated cells.

FIG. 20 shows the results of confirming the degree of encapsulation of cells according to the number of encapsulation layers.

FIGS. 21A-21C show the results of confirming a survival rate of encapsulated cells.

FIGS. 22A and 22B are the results of confirming persistence of cell encapsulation layer according to the presence or absence of a cross-linking of enzymes.

FIGS. 23A-23D are the results of confirming a response of encapsulated cells to glucose.

FIG. 24 is the results of confirming protease and physical stress resistance of encapsulated cells.

FIG. 25 is the results of confirming cytokine resistance of encapsulated cells.

FIG. 26 is a schematic diagram and the results of co-incubation of encapsulated cells and natural killer cells (NK cells).

FIG. 27 is a schematic diagram of an encapsulation technique using the modified anticoagulating hydrogel for cell encapsulation.

FIG. 28 shows schematic diagrams of the formation process of cell clusters and the encapsulation process using the modified anticoagulating hydrogel for cell encapsulation.

FIG. 29 shows the results of nuclear magnetic resonance analysis and surface potential analysis after introducing a monophenol group to each polymer constituting the modified anticoagulating hydrogel for cell encapsulation.

FIG. 30 shows the image results of the toxicity analysis of cell clusters encapsulated with the modified anticoagulating hydrogel for cell encapsulation.

FIGS. 31, 32A and 32B show the results confirming the persistence of the modified hydrogel capsules.

FIG. 33 shows the results of confirming the coagulation delay performance of the modified hydrogel capsule in plasma.

FIGS. 34A and 34B show the results of confirming the coagulation delay performance of the modified hydrogel capsule in blood.

FIG. 35 shows the results of confirming the degree of platelet adhesion of the modified hydrogel.

FIG. 36 shows the results of confirming the cross-linking ability of the sulfated polymer.

FIG. 37 shows the results of cross-validation of the introduction of monophenol groups into sulfated polymers.

FIGS. 38A and 38B show the encapsulation and coating time of the modified hydrogel.

FIG. 39 shows the results of fluorescence imaging of the degree of collagen adsorption of beads encapsulated with modified hydrogels.

FIG. 40 is a graph showing quantitative data of the fluorescence intensity of collagen adsorbed on beads encapsulated with modified hydrogels.

FIG. 41 is a photograph of a mesenteric vein where pancreatic islet cells have engrafted.

FIG. 42 is a graph of blood glucose level in the control group in the allogeneic pancreatic islet cell transplantation model.

FIG. 43 is a graph showing changes in blood glucose level and body weight in the group of modified hydrogel for cell encapsulation in the allogeneic pancreatic islet transplantation model.

### BEST MODE FOR CARRYING OUT THE INVENTION

Since the present invention may apply various modifications and have various embodiments, specific embodiments are intended to be illustrated in the drawings and described in the specification in detail. However, these descriptions are not intended to limit the present invention to the specific embodiments, but should be understood to include all modifications, equivalents, or substitutions involved in the spirit and scope of the present invention. If it is considered that the detailed descriptions of related known technologies may obscure the gist of the present invention, those descriptions will be omitted.

Hereinafter, a method for modifying and cross-linking the outermost layer of an anticoagulating hydrogel for cell encapsulation according to an embodiment of the present invention will be described in more detail.

To solve the problem of limited exchange of external substances of microbeads and the problem of reduced stability of a layer-by-layer cell coating, the present invention provides a hydrogel for cell encapsulation having an appropriate thickness and strong bonding of a coating layer to facilitate exchange of substances inside and outside the capsule. The hydrogel of the present invention can address problems of immune responses that may occur when transplanting cells into the body, such as blood coagulation reactions, since the polymer on the outermost surface is modified to provide an anticoagulant function. By introducing sulfated polymers to the outermost surface of the hydrogel, covalent bonds can be formed with the underlying layer, and particularly the instant blood-mediated inflammatory response can be effectively controlled.

Specifically, the modified anticoagulating hydrogel for cell encapsulation according to the present invention comprises a chitosan layer and a hyaluronic acid layer, wherein the chitosan layer and hyaluronic acid layer form cross-linking with each other by an enzyme, and the outermost layer layered with a sulfated polymer.

According to one embodiment, the chitosan may include chitosan or a derivative thereof. Specifically, for example, it may include glycol chitosan, and the molecular weight of the chitosan may be, for example, 15 kDa to 310 kDa, for example, 75 kDa to 310 kDa.

In addition, for example, the molecular weight of hyaluronic acid may be from 1.2 kDa to 8,000 kDa, for example from 40 kDa to 64 kDa.

The ‘cross-linked bond’ may be used synonymously with a ‘bridge bond’ or a ‘cross-linking’.

Also, it means a completely chemical bond such as a covalent bond or an ionic bond between a molecule and a molecule. For example, the chitosan and the hyaluronic acid in the present invention may form the covalent bond with each other, and may be crosslinked by an aryloxy coupling.

According to an embodiment, the chitosan and the hyaluronic acid may each independently be functionalized with phenol, catechol or quinone, and specifically, may be functionalized with, for example, phenol respectively, for example, mono-phenol.

According to one embodiment, the sulfated polymer may include one or more of chondroitin sulfate, sulfated hyaluronic acid, and heparin, and specifically may include heparin, for example.

The sulfated polymer may also have a monophenol group introduced therein, and the outermost layer of the hydrogel can be modified through the monophenol group, thereby imparting an anticoagulant effect to the hydrogel. Specifically, for example, the sulfated polymer having a monophenol group introduced may include a compound having a structure represented by chemical formulas 1 to 3.

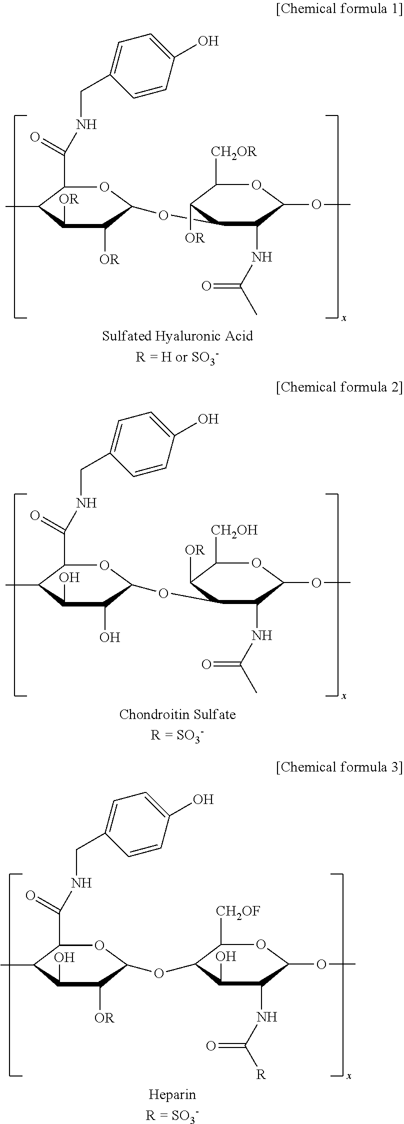

The present invention can also provide a modified anticoagulating hydrogel for cell encapsulation composition comprising the modified anticoagulating hydrogel for cell encapsulation as described above and isolated cell.

According to one embodiment, the cells to be encapsulated are not particularly limited, and one or more cells can be encapsulated with the hydrogel of the present invention. For example, it is possible to encapsulate a single type of cells or one or more types of cells. Additionally, it is possible to encapsulate individual cells or cell clusters at once, and it is also possible to encapsulate organs by coating them. For example, by encapsulating pancreatic islet cells in nano or macro units according to the present invention, the use of immunosuppressants can be minimized, and long-term and stable immune and immediate blood glucose control can be achieved evasion by prevention of antigen exposure. Additionally, specific examples of the type of cell which can be encapsulated may include one or more of β-cells, adrenal endothelial cells, muscle cells, fibroblasts, and blood cells.

According to one embodiment, the chitosan contained in the hydrogel may be in direct contact with cells. Specifically, for example, the layer closest to the cell may be a chitosan layer comprising chitosan functionalized with phenol wherein the chitosan layer forms aryloxy cross-linking by an enzymatic reaction of tyrosinase.

According to an embodiment, the modified hydrogel for cell encapsulation according to the present invention may form a film in which 1 to 10 layers, for example, 2 to 8 layers, preferably 3 to 7 layers, are layered on the outside of the cells. Specifically, for example, the composition may comprise at least two cell encapsulating layers, for example, have a structure in which the chitosan layer and the hyaluronic acid layer are each independently layered in 1 to 5 layers, for example, 1 to 3 layers alternately or crosswisely. In addition, the modified hydrogel of the present invention may have an outermost layer formed of sulfated polymers.

Compared to the cell spherical encapsulation using the alginate beads of the prior art, the encapsulation layer in the form of the thin film of the multiple layers (Layer-by-layer, LBL) according to the present invention is formed of a very thin polymer layer to allow the cells to respond rapidly to detection of glucose and secretion of insulin. Specifically, the prior art has used the alginate beads having a size of 500 microns, which limits the rapid response of the cells to detect the glucose and secrete the insulin, for example, because the glucose must move several hundred microns into the alginate beads.

Electrostatic interaction between the polymers has been conventionally applied to form a thin-film coating of the multiple layers, but it has a disadvantage in that it requires an excessively long preparation time to achieve optimal condition of the cell encapsulation.

The present invention can solve the above disadvantage by inducing fast cross-linking using an enzyme.

According to another embodiment of the present invention, it is provided a method for producing a modified anticoagulating hydrogel for cell encapsulation, comprising the steps of:
(i) adding a solution containing a functionalized chitosan and a solution containing a functionalized hyaluronic acid to form layers having a chitosan layer and a hyaluronic acid layer alternately layered, wherein the chitosan layer and the hyaluronic acid layer form aryloxy cross-linking with each other by using tyrosinase enzyme; and
(ii) adding a solution containing a functionalized sulfated polymer onto the layers obtained from step (i) to form an outermost layer, wherein the outermost layer forms aryloxy cross-linking by using tyrosinase enzyme,
wherein the sulfated polymer comprises at least one of chondroitin sulfate, sulfated hyaluronic acid, and heparin.

According to one embodiment, the chitosan, hyaluronic acid and sulfated polymer may each independently have phenol, catechol or quinone residues introduced therein, for example, phenol residues introduced therein. Introduction of the above residues can induce crosslinking between chitosan, hyaluronic acid, and sulfated polymer. For example, glycol chitosan can be functionalized by introducing monophenol moiety thereinto by treating with 4-hydroxyphenylacetic acid or 3-(4-hydroxyphenyl) propionic acid. In addition, catechin, epigallocatechin gallate (EGCG), and other polyphenol compounds can be introduced.

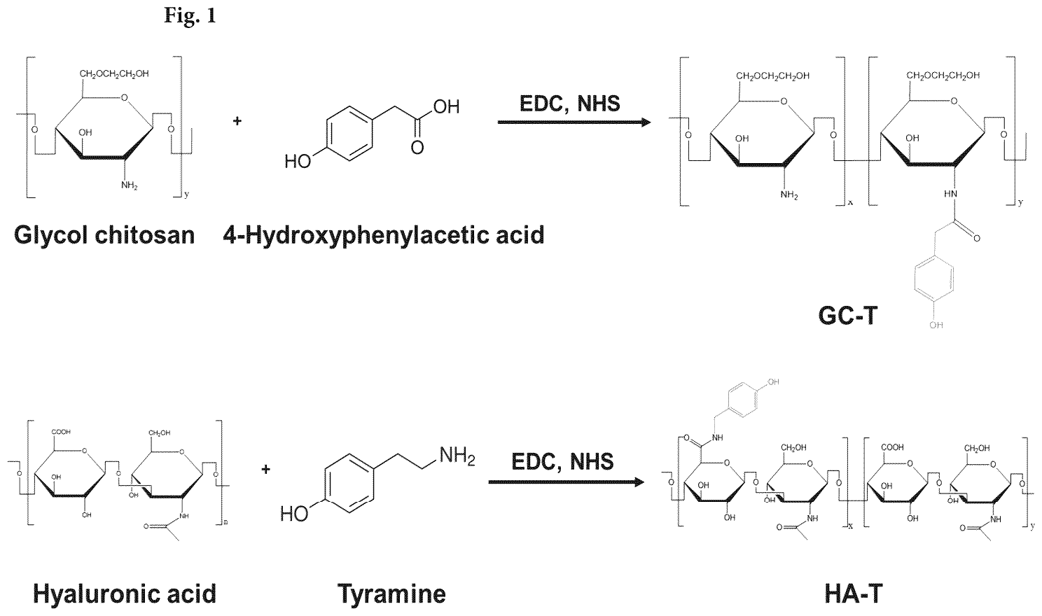

Further, the monophenol moiety may be introduced into the hyaluronic acid by treating the hyaluronic acid with at least one selected from the group consisting of tyramine and tyrosine. In addition, for example, the tyramine may be used in the form of a hydrochloride salt thereof. A process of introducing the phenol moiety according to an embodiment is shown in FIG. 1.

According to one embodiment, after the step of introducing the phenol moiety, the chitosan and the hyaluronic acid can be crosslinked by enzymatic reaction which results from addition of one or more enzymes selected from the group consisting of monophenol oxidase, diphenol oxidase and laccase to each of the chitosan and the hyaluronic acid. For example, the cross-linking may include an aryloxyl coupling.

According to one embodiment, the functionalized chitosan may be the chitosan modified with at least one aryl group, the functionalized hyaluronic acid may be the hyaluronic acid modified with at least one aryl group, and the functionalized sulfated polymer may be the sulfated polymer modified with at least one aryl group.

According to one embodiment, the functionalized chitosan may be contained in an amount of 0.01 to 5% (w/v), for example 0.1 to 1% (w/v) in the solution, the functionalized hyaluronic acid may be contained in an amount of 0.01 to 5% (w/v), for example 0.1 to 1% (w/v) in the solution, and the functionalized sulfated polymer may be contained in an amount of 0.01 to 5% (w/v), for example, 0.1 to 1% (w/v) in the solution.

According to one embodiment, the monophenol oxidase may include tyrosinase and the diphenol oxidase may include catechol oxidase.

The tyrosinase is a metalloenzyme containing a copper and is present in various plants such as a mushroom, a potato and an apple, and animals. The tyrosinase acts as a cresolase that produces o-diphenol by oxidizing the mono-phenol, and acts as a catecholase that produces o-quinone by oxidizing the produced o-diphenol again.

The catechol oxidase has a function almost similar to that of the tyrosinase. However, the catechol oxidase can act as the catecholase that produces the o-quinone, but cannot act as the cresolase that oxidizes the mono-phenol, which is another function of tyrosinase.

The enzyme of the present invention may include tyrosinase, for example, tyrosinase derived from Streptomes sp. Specifically, for example, the enzyme of the present invention may include tyrosinase derived from Streptomyces avermitilis (Streptomyces avermitilis-derived tyrosinase). The tyrosinase derived from actinomycete of the Streptomes sp. has a higher catalytic efficiency for a tyrosine moiety bound to the polymer than that of a mushroom-derived tyrosinase, and thus shows a high conversion efficiency to dopa or quinone moiety. The converted moiety is cross-linked by a covalent bond or a coordination bond, and due to a dense cross-linking reaction, non-specific cross-linked bond is eliminated to shorten the cross-linking time and show high thermal and mechanical stability. Since the present invention uses only the cross-linked protein and the cross-linking tyrosine, it is possible to overcome the technical limitation of the prior art for the toxicity of monomeric chemicals and the limited accessibility of enzymes.

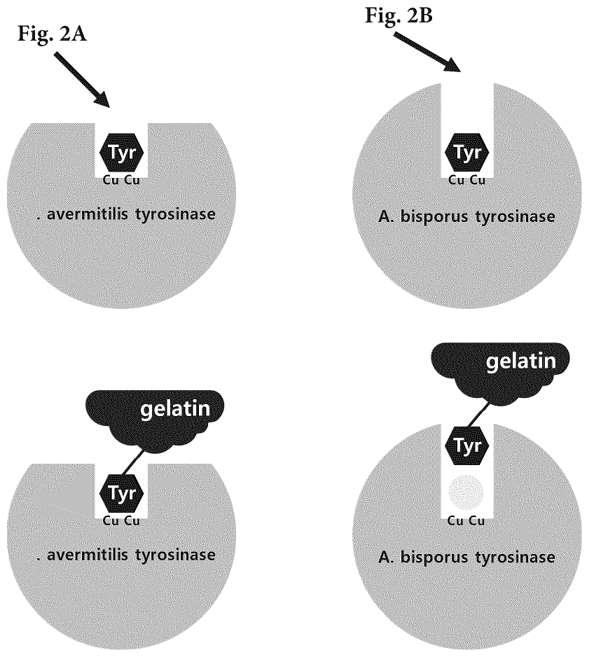

The actinomycete-derived tyrosinase is structurally different from the other species-derived tyrosinase as it has an open structure around the catalytic reaction and has very few steric hindrance factors. A structural comparison diagram of the mushroom-derived tyrosinase and the actinomycete-derived tyrosinase is shown in FIGS. 2A and 2B. FIG. 2A shows the actinomycete-derived tyrosinase, and exhibits that the steric hindrance around the entrance to which the tyrosinase accepts a substrate is small.

In contrast, FIG. 2B shows the mushroom-derived tyrosinase, and exhibits that accessibility to a tyrosine located in a protein of a large volume such as gelatin is reduced by a steric hindrance factor due to unnecessary peptides around the entrance. That is, the actinomycete-derived tyrosinase is advantageous for hydroxylation and oxidation reaction of the tyrosine with the polymer material, compared to the conventional mushroom-derived tyrosinase.

As a result, if reaction time of the conventional tyrosinase takes several hours, the tyrosinase derived from Streptomyces avermitilis can induce a quick and rapid reaction at several seconds. Therefore, in the step of adding the enzyme to crosslink the polymer, the reaction time may be 1 minute to 1 hour, for example, 1 minute to 30 minutes, or 3 minutes to 20 minutes, or 5 minutes to 15 minutes.

The phenol introduced into the chitosan and hyaluronic acid polymers can be oxidized by the enzyme as described above to form dopa or quinone, for example, o-quinone, and the dopa or quinone moiety can form a cross-linking by a coordination bond, that is, a covalent bond, to have a high stability due to a dense cross-linked structure.

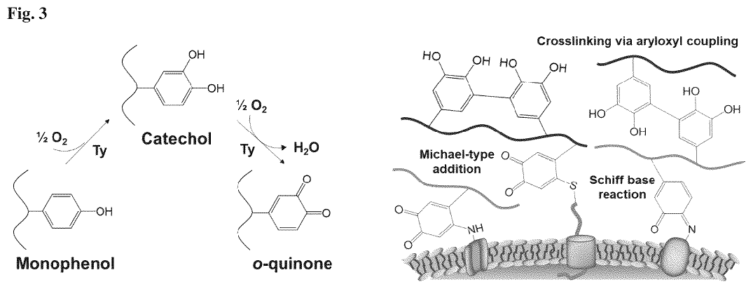

According to an embodiment of the present invention, the process of introducing the phenol group into the polymer and cross-linking it by oxidization is shown in FIG. 3. Compared with the conventional DOPA-modified macromolecules, the enzyme-based molecules according to the present invention are particularly efficient for oxidizing the phenol moiety, and can accelerate the cross-linking through a permanent covalent bond between catechol quinones, and amines, thiols and other phenol groups. In addition, since this enzyme itself, catechol, and catechol quinine-based substances do not have cytotoxicity or inflammation, a thin hydrogel can be formed on the cells using the tyramine-coupled chitosan and hyaluronic acid.

According to an embodiment, the step of adding the enzyme may include adding the tyrosinase of 0.01 to 0.5 U/ml, for example 0.01 to 0.3 U/ml, or 0.01 to 0.1 U/ml, to the solution having the chitosan in a concentration of 0.01 to 1% (w/v), for example, 0.05 to 0.5% (w/v), for example, 0.05 to 0.3% (w/v), to the solution having the hyaluronic acid in a concentration of 0.01 to 1% (w/v), for example, 0.05 to 0.5% (w/v), for example, 0.05 to 0.3% (w/v), or the solution having the sulfated polymer in a concentration of 0.01 to 5% (w/v), for example, 0.05 to 0.5% (w/v).

According to an embodiment, the step of adding the cells may include treating and immersing the cells on a membrane, and thus, for example, may seed the cells on the membrane to 1×10⁴ cells/ml to 1×10¹⁰ cells/ml. In addition, the membrane may have pores of 0.5 to 10 μm, for example 1 to 8 μm, or 2 to 5 μm. A type of the membrane may be selected, for example, one or more from the group consisting of polycarbonate (PC), cellulose, and polyvinylidene fluoride (PVDF), but is not particularly limited thereto.

According to an embodiment, the cell encapsulation layer according to the present invention may be formed in a type of the layered hydrogel nanofilm, and have a thickness of 50 to 500 nm, for example 100 to 300 nm, or 100 to 200 nm, wherein the thickness may be appropriately adjusted depending on the use, the application environment, and the like.

According to an embodiment, the hydrogel composition for cell encapsulation may be imaged by introducing a labeling material such as a fluorescent molecule or a light emitting molecule. In addition, drugs, growth factors, etc. may be introduced and delivered locally to an application target.

For example, the cell encapsulation layer composition may be imaged by introducing a labeling material, for example, a chromogenic enzyme such as peroxidase, alkaline phosphatase, a radioactive isotope, a colloid, phycoerythrin (PE), fluorescein carboxylic acid (FCA), HRP, TAMRA, poly L-Lysine-fluorescein isothiocyanate (FITC), rhodamine-B isocyanate (RITC), rhodamine, cyanine, Texas Red, fluorescein, phycoerythrin, quantum dots, etc. Further, for example, an antibody epitope, a substrate, a cofactor, an inhibitor or an affinity ligand may be introduced.

As described above, since the hydrogel for cell encapsulation according to the present invention forms a covalent bond between the polymers constituting the capsule by simultaneously applying an electrostatic attraction and an enzyme-mediated chemical reaction, it is not easily damaged by physical stimuli such as a pressure, a blood flow in the body, etc., upon injecting the encapsulated cells, thereby improving a stability and a persistence. In addition, regardless of the type of the cells, viable cells may be encapsulated in a single cell unit or in two or more types of multicellular units.

Further, it is possible improve the survival rate of cell clusters as well as biopharmaceuticals such as antibody proteins in the body. Since the encapsulation layer of the present invention can protect the cells from cytokine attack, it makes possible it to encapsulate by coating an organ, and can be applied as a platform for organ transplantation or a cell therapeutic agent, so that it is possible to prevent physical contact with immune cells in the body and to alleviate immune rejection. In addition, by modifying the surface using sulfated polymers, the degree of blood coagulation and platelet adhesion, which are blood-mediated inflammatory reactions, can be controlled, thereby blocking the immune response and suppressing the coagulation reaction in the body upon blood contact to improve the cell survival rate.

Therefore, the present invention may be easily applied to the transplantation of isolated cells, and is particularly suitable for application to vascular injection.

Hereinafter, Examples of the present invention will be described in detail so that those of ordinary skill in the art can easily carry out the present invention. However, the present invention may be embodied in several different forms and is not limited to the Examples described herein.

### Example 1

Introduction of Phenol Moiety into Glycol Chitosan and Hyaluronic Acid

A monophenol moiety was introduced into each of glycol chitosan (GC) and hyaluronic acid (HA) polymers to synthesize chitosan-monophenol (GC-T) and hyaluronic acid-monophenol (HA-T). After dissolving 200 mg of the glycol chitosan in 10 ml of 0.1M pH 4.7 MES buffer, 160.82 mg of 4-hydroxyphenyl acetic acid (HPA) was dissolved in 10 ml of the MES buffer, and 202.64 mg of EDC and 114.76 mg of NHS was added to the solution and stirred for 5 minutes. Subsequently, the two solutions were mixed and reacted overnight at a room temperature. The solutions were then dialyzed against a distilled water for 72 hours and freeze-dried for at least 72 hours. After dissolving 200 mg of the hyaluronic acid in 20 ml of 0.1M pH 4.7 MES buffer, 197.452 mg of the EDC and 111.822 mg of the NHS were added and stirred for 5 minutes. Subsequently, 178.85 mg of tyramine hydrochloride was added and reacted at a room temperature overnight. The solution was then dialyzed against a distilled water for 72 hours and freeze-dried for at least 72 hours.

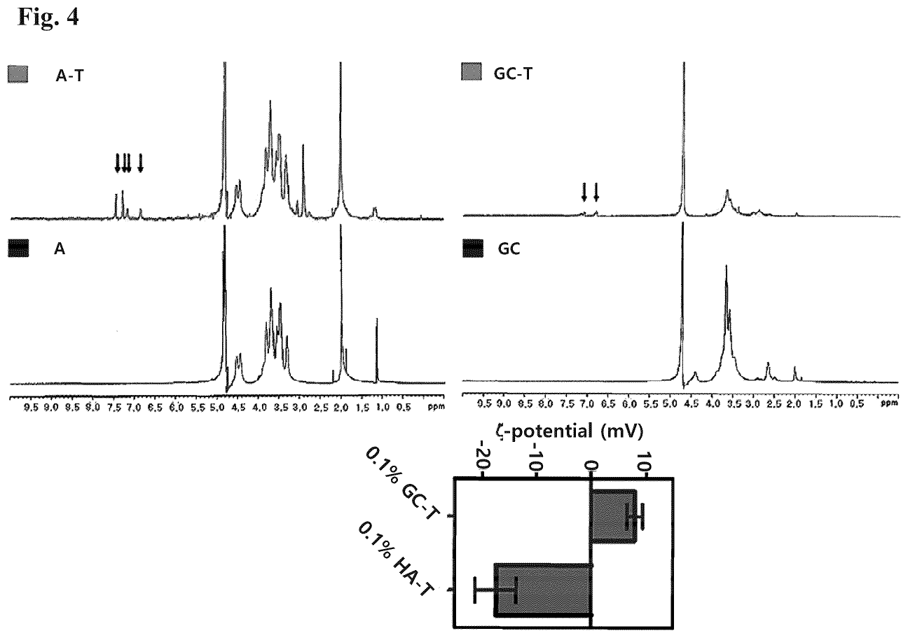

It was confirmed by a nuclear magnetic resonance spectroscopy (NMR Spectroscopy) that the monophenol moiety was introduced into the glycol chitosan and hyaluronic acid polymers, and the results were shown in FIG. 4.

Further, in order to synthesize a fluorescent polymer, 1 ml of 10 mg/ml RITC (Rhodamine B isothiocyanate/Sigma-Aldrich) and FA (fluoresceinamine isomer I/Sigma-Aldrich) solution dissolved in N, N-dimethylformamide (DMF) was added to a reaction solution of the GC-T and the HA-T, respectively, to synthesize GC-T-RITC and HA-T-FA.

Reaction of Tyrosinase with GC-T and HA-T

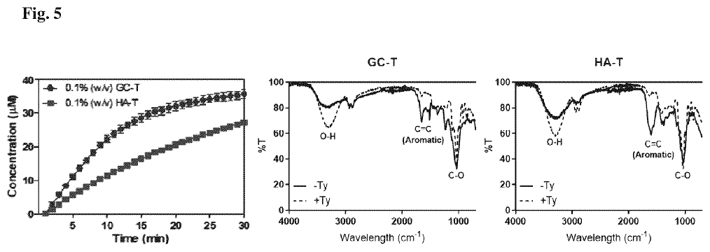

Tyrosinase was used as an actinomycete derived from Streptomyces avermitilis (SA). 2.5 μM of SA-derived tyrosinase (SA-Ty), 5 μM of CuSO₄ and a substrate were prepared in a total volume of 200 μl of 50 mM Tris-HCl buffer having pH 8.0. The substrate is 200 μM of either GC-T 1% (w/v), HA-T 1% (w/v) or L-tyrosine. Using a microplate reader (Infinite M200 PRO, TECAN, Switzerland), an absorbance was measured at 37° C. at 475 nm (ε dopachrome=3600 M⁻¹ cm⁻¹) every 1 min for 30 min. The results were shown in FIG. 5, wherein the initial reaction rate of SA-Ty is defined as a slope of the concentration and the reaction time of the product. Enzyme activity is also expressed in units per ml (U/ml), in which one unit (U) is an amount of the SA-Ty catalyzing a L-tyrosine reaction at 1 μmol per minute.

### Cell Encapsulation

The freeze-dried GC-T and HA-T were dissolved in PBS and 0.1% acetic acid at each 10 mg/ml. After complete dissolution, each solution was diluted 10-fold with the PBS to prepare it in a final concentration of 1 mg/ml, and filtered through a sterile 0.2 μm membrane. The collected Jurkat cells were washed twice with the PBS and prepared at a density of 1×10⁷ per 1 ml of the PBS. 100 μl of the cell suspension was seeded on a 3.0 μm polycarbonate membrane (Transwell® 6.5 mm insert, 24-well plate, Corning). Cell encapsulation was performed by repeating the following method using GC-T solution for odd-numbered layers of layers 1, 3 and 5, and HA-T solution for even-numbered layers of layers 2, 4 and 6. After adding 600 μl of 1 mg/ml GC-T or HA-T solution and 0.05 U/ml SA-Ty to the 24-well plate, the cell-seeded inserts were immersed at a room temperature (RT) for 10 min.

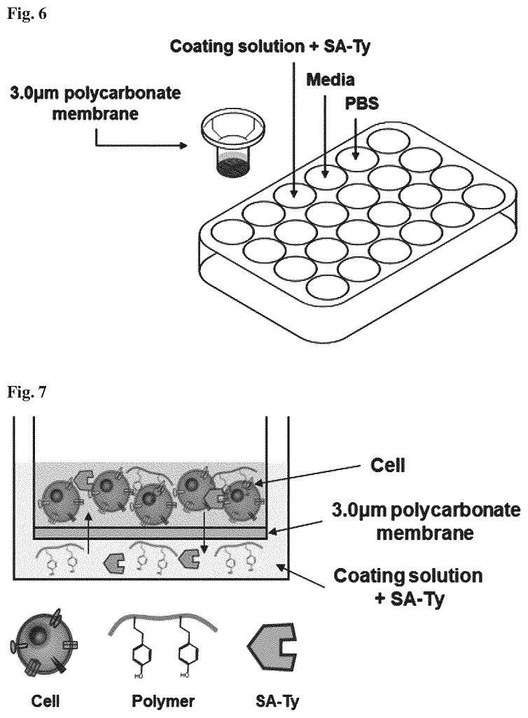

During the incubation, the plate was tapped every 2 min. After 10 min, the inserts in a shaking incubator were transferred to a well into which 500 μl of a medium (Roswell Park Memorial Institute 1640 (Gibco) containing 10% FBS (Corning)) was added for 30 seconds, and were transferred to a well into which 500 μl of the PBS was added for 1 min. Thereafter, the same method as described above was applied to the HA-T and GC-T to produce a final layer, and the cells were dispersed in a cell incubation medium (Roswell Park Memorial Institute 1640 (Gibco) containing 10% FBS (Corning)). A schematic diagram of the above process was shown in FIG. 6. Further, a specific encapsulation principle was schematically illustrated in FIG. 7.

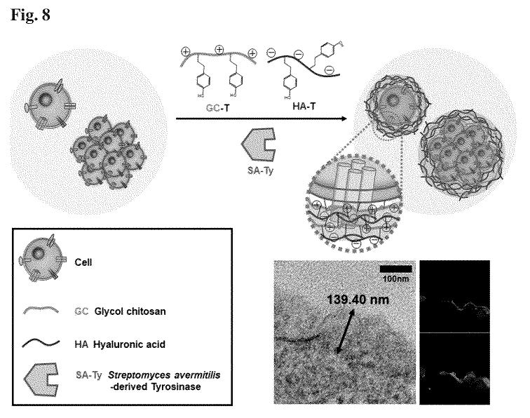

An appearance of the encapsulated cells prepared by the above method was shown in FIG. 8. FIG. 8 is a view of the encapsulated cells in which a hydrogel nanofilm is layered in 6 layers. A visualization photograph of a red fluorescence (RITC)-labeled glycol chitosan layer and a green fluorescence (FA)-labeled hyaluronic acid layer can be confirmed from FIG. 8. In addition, it is confirmed that a thickness of the hydrogel nanofilm layered on the outside of the cells was 139.4 nm.

### Confirmation of Cell Clumping Depending on GC-T Concentration

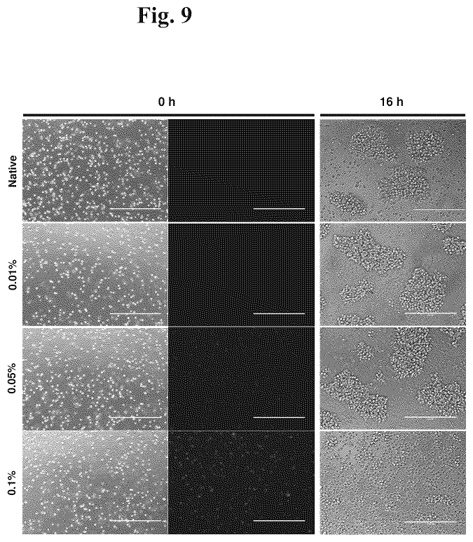

By varying a concentration of the glycol chitosan-monophenol (GC-T), the Jurkat cells having cell clumping property were encapsulated, and the results were shown in FIG. 9. When the cells were observed after 16 hours, it was confirmed that the cell clumping phenomenon was shown at a concentration of 0.05% or less, whereas a repulsion phenomenon between the cells occurred at a concentration of 0.1% or more. This is a phenomenon caused due to a charge of the glycol chitosan-monophenol (GC-T) encapsulated on the cell surface.

### Experimental Example 1: Confirmation of Cytotoxicity of Actinomycete-Derived Tyrosinase

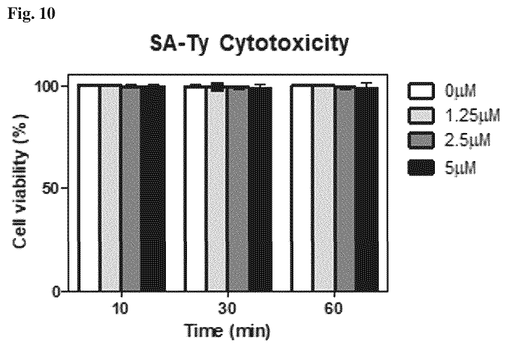

After Streptomyces avermitilis (SA)-derived tyrosinase (SA-Ty) was incubated by mixing it in media at a concentration of 0 to 5 μM for 10, 30, and 60 minutes, respectively, a survival rate of the cells was observed by a Live/Dead assay. The results were shown in FIG. 10 that indicates the survival rate of 97% or more among the overall cells. Therefore, it was confirmed that there was no toxicity when the cells were treated at a maximum of 5 μM for 60 minutes.

### Experimental Example 2: Optimization of Cell Encapsulation Condition

In order to optimize coating of a cell encapsulation hydrogel film, a relationship between a SA-Ty concentration, a reaction time and a polymer concentration was analyzed. The degree of coating was checked by measuring fluorescence intensity of GC-T-RITC and HA-T-FA. MIN6 β cells seeded in a 96-well tissue incubation plate were incubated with 0.05 U/ml of SA-Ty in 0.1% of GC-T-RITC solution or 0.1% of HA-T-FA solution for 10 minutes. After washing with PBS, fluorescence intensity was measured at two different areas; among the measurement areas, one is λex=543 nm/λem=580 nm (RITC), and the other is λex=495 nm/λem=525 nm (FA). For Native (N), the MIN6 β cells, which were subjected to the same procedure using the PBS instead of the coating solution, were used.

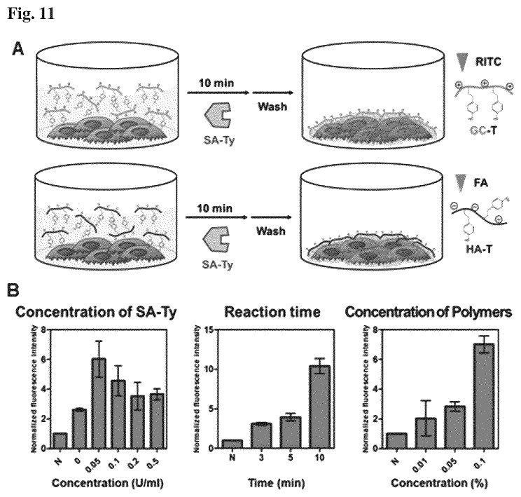

The results are shown in FIG. 11.

The highest RITC intensity was shown at a concentration of 0.05 U/ml of the SA-Ty, a reaction time of 10 minutes, and a concentration of 0.1% of the chitosan polymer. When the SA-Ty concentration was 0.05 U/ml and the chitosan polymer concentration was 0.1%, a coating level of the GC-T-RITC increased with longer the reaction time.

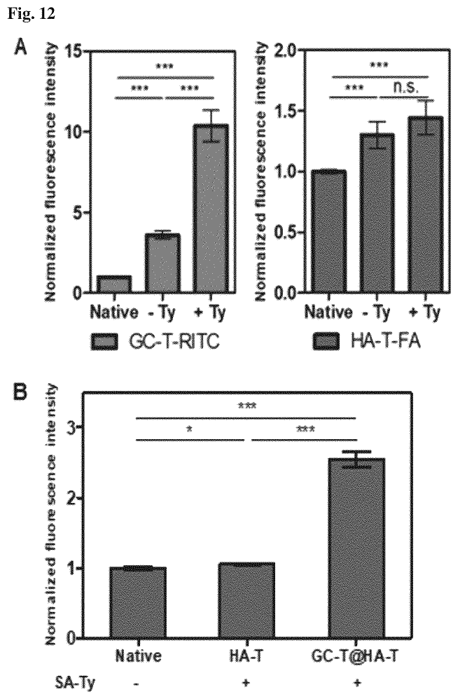

Further, a coating effect according to the presence of the SA-Ty in the GC-T-RITC and HA-T-FA solutions was investigated, respectively. In addition, an influence of the coating order was analyzed by comparing the cells coated with the single HA-T film to the cells coated with the HA-T film on the cells first coated with the GC-T film. The results were shown in FIG. 12. According to the presence of the SA-Ty, positively charged GC-T-RITC showed a difference of 2.9 times, whereas negatively charged HA-T-FA showed little difference in the degree of coating. In addition, the FA intensity was increased by 2.4 times when the HA-T film was coated on the GC-T film-coated cells.

### Experimental Example 3: Confirmation of Cell Encapsulation

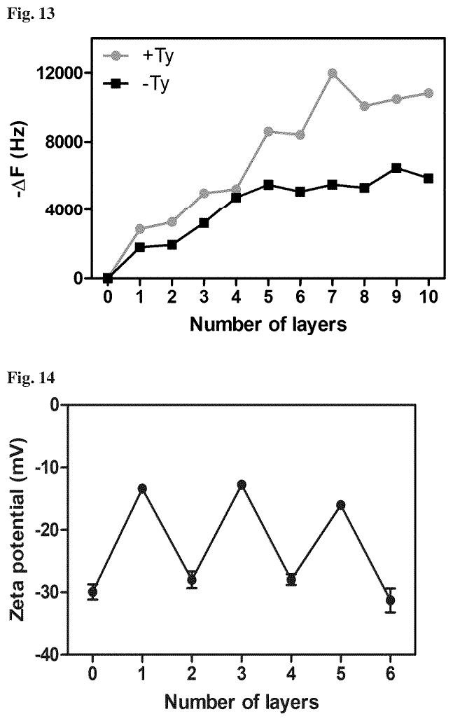

A Quartz Crystal microbalance method was used to confirm whether a hydrogel nanofilm was formed. A GC-T/HA-T layer was deposited using a Cr/Au (chromium/gold) crystal (5 MHz, 1 inch in a diameter, AT-cut, plane-plane). Prior to the deposition, the crystal was treated with a piranha solution (H₂SO₄: H₂O₂-3:1) for 5 min and an oxygen plasma for 5 min to clean the surface thereof and set a negative charge. Next, the crystal was immersed in 0.1% of the GC-T solution with 0.05 U/ml of the SA-Ty. After 10 min, the electrode was washed twice with PBS for 1 min. To remove the remaining PBS, the crystal was dried with a blower. A multilayered thin film (layer-by-layer (LBL)) deposition was performed with 0.1% of the HA-T solution until a total of five double layers were layered. The crystal on which the hydrogel film was deposited was analyzed for each layer by the quartz crystal microbalance (QCM, QCM200, Stanford Research Systems, USA) method, and the results were shown in FIG. 13.

Further, encapsulation of the cells was demonstrated.

### Zeta Potential

The Cells were fixed with 4% paraformaldehyde (PFA) for 10 minutes, and prepared in a density of 1×10⁶ cells per 1 ml of the PBS. Zeta potentials of native cells or encapsulated cells were measured with Nano ZS (Malvern Instruments, Germany) and were shown in FIG. 14.

### Fluorescence Activated Cell Sorter (FACS)

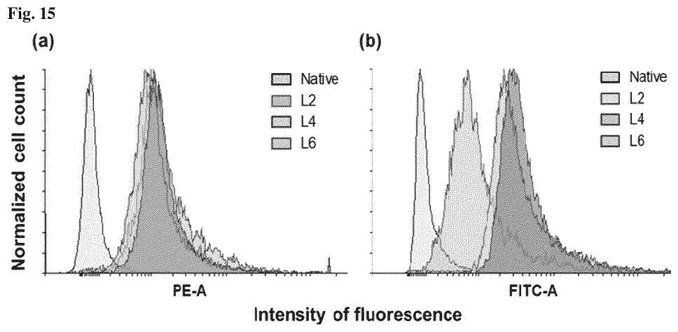

The cells were encapsulated with the GC-T-RITC and the HA-T-FA instead of the GC-T and the HA-T. After fixation, 1×10⁶ cells per 500 μl of the PBS were prepared. Fluorescence of the RITC and the FA was measured by a flow cytometry (FACS Aria II, BD Biosciences, USA) using a laser with wavelengths of 488 nm and 633 nm. The monolayered Cells encapsulated with the GC-T-RITC and the HA-T-FA were used as positive controls for gating, and the results were shown in FIG. 15.

### Confocal Laser Scanning Microscope (CLSM)

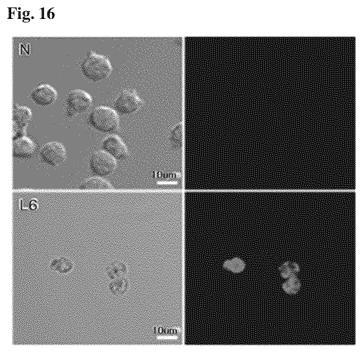

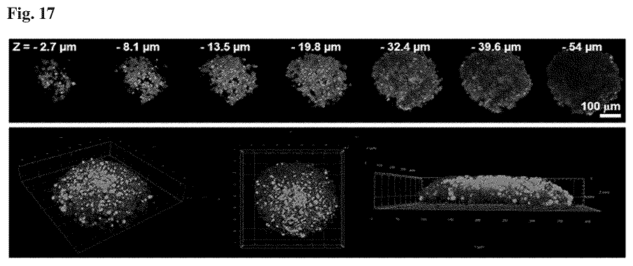

The Single cell or the B-cell spheroids (pancreatic cells) were encapsulated with the GC-T-RITC and the HA-T-FA, respectively, and then fixed with 4% PFA at a room temperature (RT) for 15 min. The spheroids in the form of an ellipsoid revolution were placed in a 20 mm confocal dish. They were imaged through a confocal microscope (LSM 780, Carl Zeiss, Germany). The results for the single cell were shown in FIG. 16, and the results for the β-cell spheroids were shown in FIG. 17. Since fluorescence appeared mainly on the outer surface of the encapsulated spheroids, it was confirmed that the hydrogel nanofilm was uniformly covered on the surface of macromolecules.

### Scanning Electron Microscope (SEM)

SEM (JSM−6701F, JEOL) images were analyzed to observe the surface of the encapsulated β-cell spheroids.

For preparation of samples for the SEM analysis, the spheroids were fixed with 2.5% glutaraldehyde solution for 4 hours, and dehydrated with ethanol. Then, they were dried through hexamethyldisilazane, and observed by carrying out platinum coating for 120 seconds.

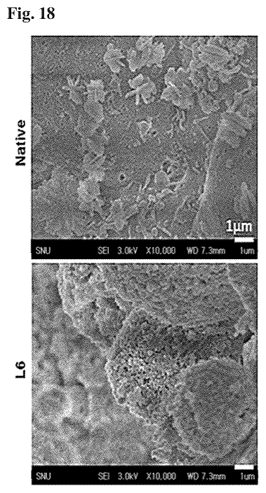

The results were shown in FIG. 18. When the GC-Tis uniformly coated on the Jurkat cells, the cells repel each other and are coated with a uniform cover of the polymer. It was confirmed that the cross-linked polymer was evenly layered on the surface of the encapsulated spheroids. The surface of the encapsulation layers formed a mesh structure.

On the contrary, the native spheroids had a smooth surface that is not coated with the polymer.

### Transmission Electron Microscope (TEM)

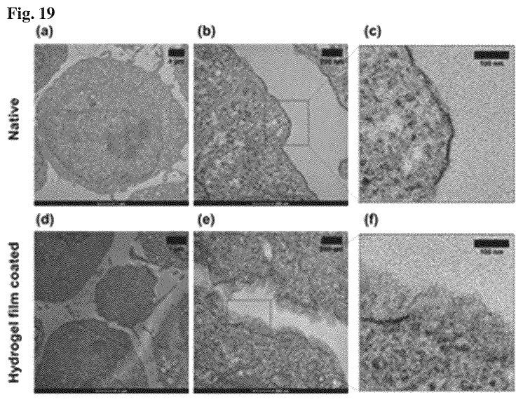

TEM (Talos L120C, 120 kV, FEI, Czech) images were analyzed to confirm whether the hydrogel is formed in a thickness of nanometer (nm) on the cell surface. For preparation of TEM samples, the native cells and the encapsulated cells were fixed with a Karnovsky's fixing solution. The cells were treated with 1% osmium tetroxide in a cacodylate buffer for 1 hour, and then treated with 0.5% uranyl acetate at 4° C. for one day. After dehydration with ethanol, the samples were embedded in a Spurr resin. The specimens were cut using an ultramicrotome (EM UC7, Leica, Germany), and the analysis results were shown in FIG. 19.

### Fluorescence Microscope

Fluorescence images were observed to confirm the degree of cell encapsulation for each layer number of the encapsulated β-cell spheroids.

When the β-cell spheroids were encapsulated with the GC-T-RITC, the HA-T-FA, it was confirmed by imaging with a fluorescence microscope (EVOS® Cell Imaging Systems, Thermo Fisher Scientific) that the fluorescence intensity increased as the number of layers increased.

The results were shown in FIG. 20.

### Experimental Example 4: Confirmation of Survival Rate of Encapsulated Cells

In order to confirm a survival rate of the encapsulated cells, the cells were stained and analyzed with Live/Dead® Viability/Cytotoxicity Kit containing calcein-AM and ethidium homodimer-1 (EthD-1).

The calcein-AM can be delivered to neighboring cells only through cell tunnels formed by direct contact between the cells. The calcein-AM, which has passed through the cell membrane, loses membrane permeability to stay in the cells as a bond of acetoxymethyl ester is broken by esterase in the cells and separated to calcein. Since an amount of green fluorescence measured with a microscope, a fluorometer, a flow cytometry, etc. is determined by the membrane permeability of viable cells and activity of the esterase, it reflects activity of the cells as it is.

The EthD-1 only enters dead cells having the cell membrane damaged, and emits red fluorescence by a bond to nucleic acids.

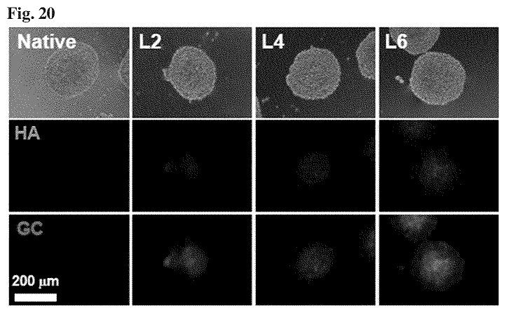

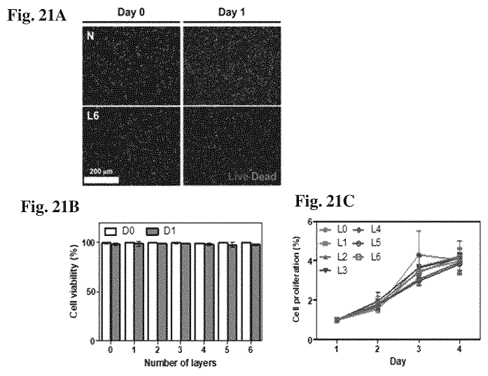

After the stained cells were imaged with a fluorescence microscope (EVOS® Cell Imaging Systems, Thermo Fisher Scientific), the cells were counted in four separate fields to calculate a percentage of the viable cells. For proliferation, cell metabolism was measured for 3 or 4 days at intervals of 24 hours using an alamarBlue reagent. The measured fluorescence of the reagent was normalized to the value of the first day. The results were shown in FIGS. 21A-21C. FIG. 21A is a photograph visualizing a survival rate of the unencapsulated Jurkat cells and the Jurkat cells encapsulated in 6 layers according to Example 1. FIG. 21B is a graph showing a survival rate of the cells according to the number of encapsulation layers of the hydrogel nanofilm. FIG. 21C is a graph showing a proliferation rate of the cells according to the number of encapsulation layers and time.

### Experimental Example 5: Confirmation of Persistence of Encapsulation Layers

In order to confirm persistence of the cell encapsulation layers according to the presence or absence of crosslinking of an enzyme, fluorescence intensity of the encapsulation layers over time was checked.

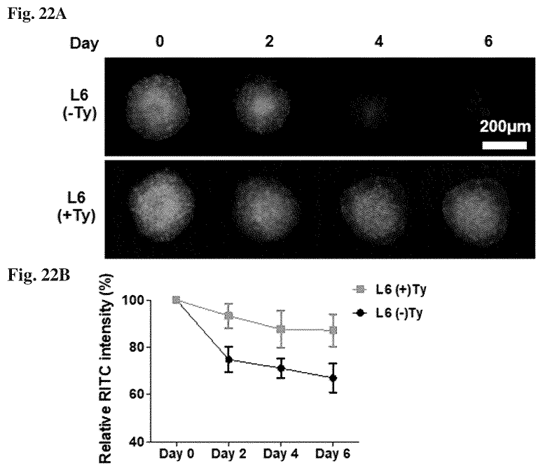

The β-cell spheroids were encapsulated using the GC-T-RITC and the HA-T-FA, and the persistence of the encapsulation layers with or without use (−Ty or +Ty) of the enzyme during encapsulation was imaged by a fluorescence microscopy (EVOS® Cell Imaging Systems, Thermo Fisher Scientific). The results were shown in FIG. 22A. It was confirmed that when the enzyme was used (+Ty), the fluorescence was maintained until 6 days after the incubation of the cells outside the body.

Further, the cell layers in a 2D state were encapsulated using the GC-T-RITC and the HA-T, and likewise, the persistence of the encapsulation layers with or without use of the enzyme was measured every 24 hours with excitation at 543 nm and emission at 580 nm, using a microplate reader (Infinite M200 PRO, TECAN, Switzerland). The results were shown in FIG. 22B. It was confirmed that the encapsulated cells were inhibited from dissociation even at 48 hours.

### Experimental Example 6: Confirmation of Response of Encapsulated Cells to Glucose

Insulin ELISA (enzyme-linked immunosorbent assay) was performed to check an insulin secretory ability and a relative gene expression according to the number of layers of the encapsulated cells. In the encapsulation layers of the present invention, low molecules such as glucose, amino acid, and insulin can freely diffuse into and out of the cells. The L6 encapsulation layers show a low diffusion rate for both the FITC-dextran of 20 kDa and 70 kDa, but does not interfere with the insulin secretion at 5.8 kDa.

An amount of the insulin secretion was measured by the ELISA of β-cell spheroids encapsulated using the GC-T and the HA-T after 1 day and 7 days, respectively.

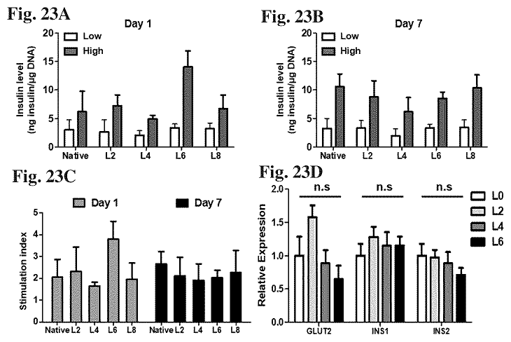

The results of checking insulin levels in the hypoglycemic and hyperglycemic solutions were shown in FIGS. 23A and 23B.

Further, in order to analyze an amount of the insulin secretion, the encapsulated β-cell spheroid samples were incubated in 3.3 mM low-concentration glucose (D-Glucose) and 20 mM high-concentration glucose solutions for 2 hours, respectively, to obtain a supernatant thereof. Thereafter, a concentration of the insulin was analyzed using a mouse insulin ELISA (80INSMS-E01, ALPCO) kit.

Glucose sensitivity was investigated from a value obtained by dividing an amount of the insulin secretion in the high-concentration glucose solution by an amount of the insulin secretion in the low-concentration glucose solution, and the result were shown in FIG. 23C.

Further, a polymerase chain reaction was performed to compare expression levels of GLUT-2, INS-1, and INS-2 as glucose and insulin-related genes. The spheroid samples were transferred to a tube containing 500 μl of a trizol solution, and 100 μl of a chloroform solution was added thereto, followed by centrifugation at the conditions of 21,055×g, 20 minutes and 4° C. After obtaining a transparent aqueous layer, 250 μl of isopropanol was added and centrifuged at the conditions of 21,055×g, 20 minutes and 4° C. After removing a supernatant, the samples were washed with 500 μl of 75% ethanol, and the precipitated samples were dissolved in a distilled water. After denaturation at 60° C. for 10 minutes, cDNA was prepared using EZ066M (Enzynomics, Korea) kit.

The expression levels of GLUT-2, INS-1 and INS-2 genes were analyzed using StepOnePlus™ Real-Time PCR system (Applied Biosystems) equipment and SYBR Green PCR Mastermix.

As a result, a graph showing changes in every number of layers of the β-cell-related genes GLUT-2, INS-1 and INS-2 involved in the insulin synthesis and secretion was shown in FIG. 23D.

### Experimental Example 7: Confirmation of Protease and Physical Stress Resistance of Encapsulated Cells

Depending on the presence or absence of encapsulation of the B-cell spheroids, they were incubated in a solution of 0.05% Trypsin as the protease for 30 minutes, respectively, followed by light pipetting to observe the degree of degradation over time.

Further, depending on the presence or absence of encapsulation of the B-cell spheroids, they were incubated in a solution of a 10 mg/ml collagenase type 2 as the protease up to 18 hours in maximum to observe the degree of degradation.

In addition, the native spheroid group and the encapsulated β-cell spheroid group were centrifuged at the conditions of 80×g and 1073×g for 5 minutes, respectively, to check the degree of resistance to physical stress (centrifugation) depending on the presence or absence of encapsulation of the β-cell spheroids.

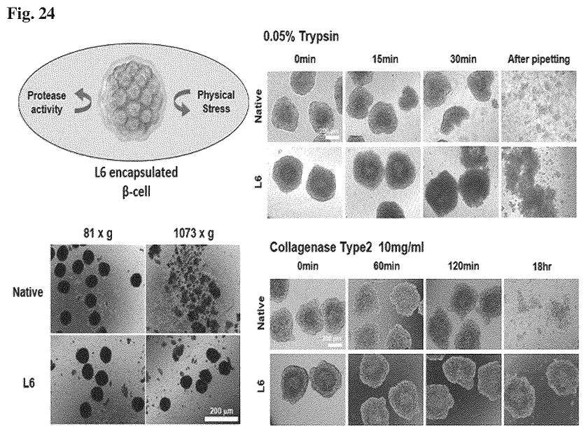

The results were shown in FIG. 24.

The native spheroid group treated with the trypsin or the collagenase type 2 was collapsed and dissociated easily by light pipetting, whereas the encapsulated spheroid group showed little separation of the cells and maintained structure thereof.

Also, with respect to the physical stress, both the native spheroid group and the encapsulated spheroid group maintained their spherical shapes at a low centrifugal speed, but the native spheroid group was collapsed at a high centrifugal speed. Contrary to this, it was confirmed that the L6 encapsulated spheroid group had a persistence due to withstanding a high pressure.

### Experimental Example 8: Confirmation of Resistance of Encapsulated Cells to Cytokine

Resistance of the Encapsulated β-Cell Spheroids to a Cytokine was Checked.

TNF-α, an inflammation-inducing cytokine, which induces apoptosis of β-cells, was analyzed through PCR to determine whether the encapsulation layers block access of the TNF-α. After 48 hours under an incubating condition of adding the TNF-α in a concentration of 10 nM, the expression level of apoptosis-related genes was compared in the native spheroid group and the encapsulated spheroid group through the PCR. The PCR process was as described above, and p53 and Caspase-3 were selected as the genes.

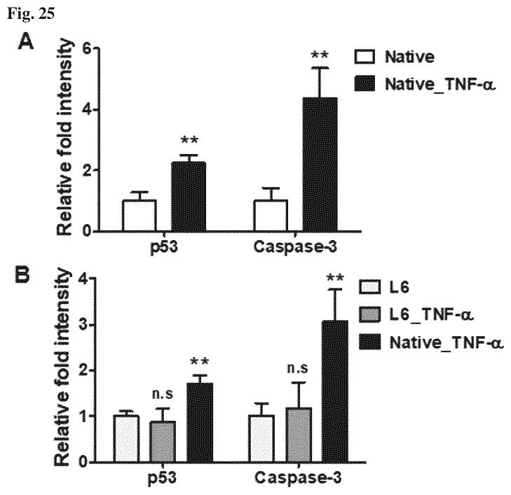

The results were shown in FIG. 25. It was confirmed that the encapsulated spheroid group did not respond to the TNF-α treatment as it protected an attack of the cytokine. These results show that the cytokines having a molecular weight close to 20 kDa cannot diffuse through the L6 encapsulated cells having the six-layered hydrogel nanofilm.

Contrary to this, the native spheroid group indicated that the cell genes such as the p53 and the Caspase-3 involved in the cell cycle and the apoptosis were significantly increased.

### Experimental Example 9: Confirmation of Blocking of Encapsulated Cells Against Immune Cells

In order to check whether the encapsulated cells block immune cells, the encapsulated β-cell spheroids and the natural killer cells (NK92c cells) were co-incubated. By cell-cell contact, it was checked whether the encapsulation layers block contact of the immune cells through the co-incubation with the natural killer cells that attack the B-cells.

The β-cell spheroid samples and the natural killer cells (NK-92 cells) were fluorescently stained using CellTracker™ Green (Invitrogen) and CellTrace Far-red (Invitrogen), respectively, and were incubated and imaged using Chamlide TC incubator system (Live-cell Instrument, Korea).

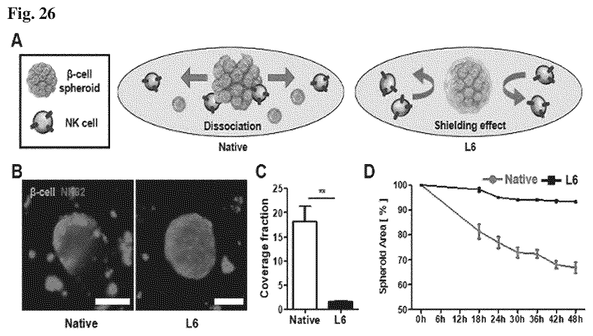

The results were shown in FIG. 26. It was confirmed that the encapsulated β-cell spheroids had little contact with the natural killer cells and a size of the spheroids was also maintained. In contrast, it was confirmed that the native spheroids had high contact with the natural killer cells and an area of the spheroids was rapidly reduced.

### Example 2: Preparation of Modified Anticoagulating Hydrogel for Cell Encapsulation

Referring to Example 1, 200 mg of glycol chitosan was dissolved in 10 ml of 0.1 M pH 4.7 MES buffer, and then to increase the monophenol group substitution rate, 1078.8 mg of 4-(4,6-dimethoxy-1,3,5-triazin-2-yl)-4-methyl-morpholinium chloride (DMTMM) was used instead of EDC/NHS to synthesize the polymer. In addition, 593 mg of 4-hydroxyphenylacetic acid (HPA) was dissolved in 10 ml of MES buffer and 366 mg of tyramine hydrochloride was added to increase the synthetic substitution rate. 200 mg of hyaluronic acid was dissolved in 20 ml of 0.1 M MES buffer, pH 4.7 and stirred for 5 min. Then, 366.2 mg of tyramine hydrochloride was added.

Referring to Example 1, a modified hydrogel for cell encapsulation was prepared as follows. 1 mg/ml (=0.1 wt %) of sulfated hyaluronic acid, 1 mg/ml (=0.1 wt %) of chondroitin sulfate, or 1 mg/ml (=0.1 wt %) of heparin as sulfated polymer were used to introduce sulfated polymers into the outermost layer. Specifically, 200 mg of chondroitin sulfate, 161.3 mg of EDC, 96.8 mg of NHS, and 292.2 mg of tyramine hydrochloride were mixed. 200 mg of heparin, 115.42 mg of EDC, 69.3 mg of NHS, and 209.1 mg of tyramine hydrochloride were mixed. 200 mg of sulfated hyaluronic acid was synthesized under the same condition as hyaluronic acid.

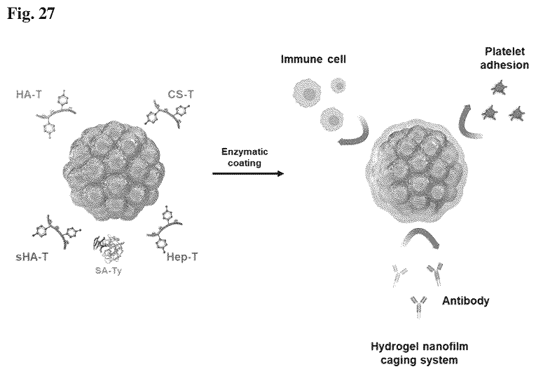

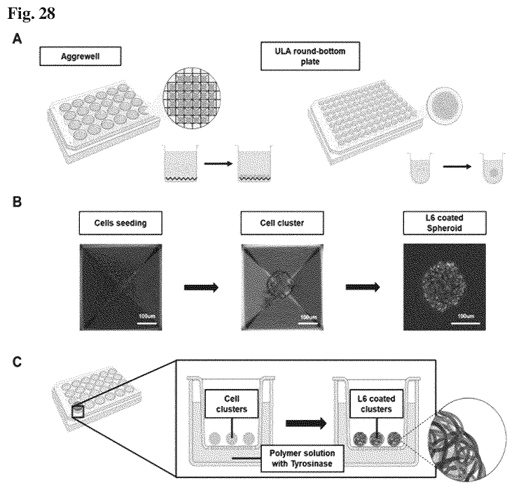

In the final step of the process of layering, the solution was replaced with a sulfated polymer solution, and the outermost layer was formed by the enzyme treatment. The reaction step of tyrosinase in Example 1 was carried out, and the substrate of the solution for the last layer was replaced with 0.1% (w/v) of sulfated polymer. Specifically, the 1st, 3rd, and 5th layers are composed of glycol chitosan layers, the 2nd and 4th layers are composed of hyaluronic acid layers, and the 6th layer is composed of an outermost layer of sulfated polymer. An example of a schematic diagram of an encapsulation using the anticoagulating hydrogel for cell encapsulation modified with sulfated polymers is shown in FIG. 27. In addition, an example of a schematic diagram of the fabrication of cell clusters and the encapsulation process using the modified anticoagulating hydrogel for cell encapsulation is illustrated in FIG. 28.

### Experimental Example 10: Nuclear Magnetic Resonance (NMR) and Surface Potential Analysis

H-NMR analysis was used to analyze the conjugation ratio of monophenol groups to optimize the synthetic method, and FT-IR spectra before and after cross-linking were compared to analyze the enzymatic cross-linking ability of sulfated polymers with monophenol groups introduced therein.

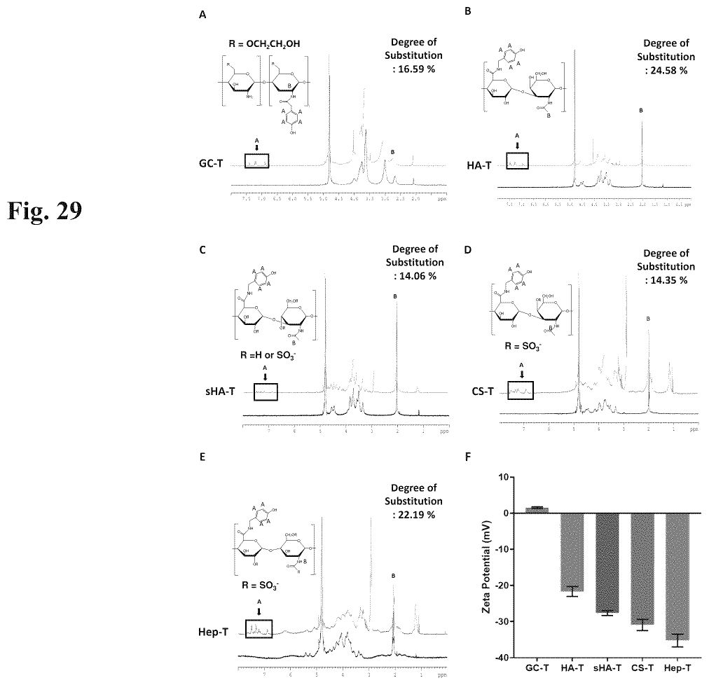

Monophenol groups were introduced into each of the polymers of glycol chitosan, hyaluronic acid, sulfated hyaluronic acid, chondroitin sulfate, and heparin, which constitute the modified anticoagulating hydrogel for cell encapsulation according to Example 2, and then nuclear magnetic resonance analysis and surface potential analysis were carried out. The surface potential analysis method was the same as the zeta potential analysis method of Experimental Example 3. The results are shown in FIG. 29.

To enhance the efficiency of layer-by-layer coating of polymers to the cell surface based on electrostatic attraction, polymers with stronger electrostatic attraction are required. From the measurement results of Zeta potential (surface potential) of sulfated polymers, it was confirmed that heparin had the highest electrostatic attraction with positively charged polymers, among hyaluronic acid, sulfated hyaluronic acid, chondroitin sulfate, and heparin.

### Experimental Example 11: Toxicity Analysis of Cell Clusters Encapsulated with Modified Anticoagulating Hydrogel for Cell Encapsulation

Immortal beta cell line MIN6 cells were formed into spheroids and encapsulated using layer-by-layer enzymatic cross-linking of glycol chitosan and sulfated polymer. Cells were added immediately after addition of enzyme to the sulfated polymer solution.

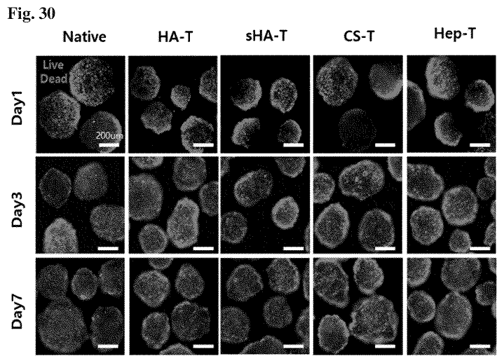

Microscopic images were observed before and after the encapsulation process to examine the surface changes of spheroids, and Live/Dead assay analysis with CalceinAM and Ethidium Homodimer was performed. The results are shown in FIG. 30, and the surface of the encapsulated cell spheroids is coated with a nanometer-thick modified hydrogel, so that the capsule layer is not visible to the naked eye. It was confirmed that the dead cells were maintained at a similar level in cell damage or morphology compared to the non-encapsulated group and that there was no effect on cell survival for up to one week.

### Experimental Example 12: Persistence of Modified Hydrogel Capsules

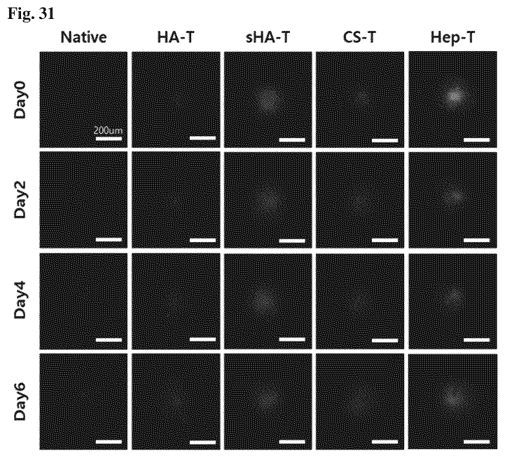

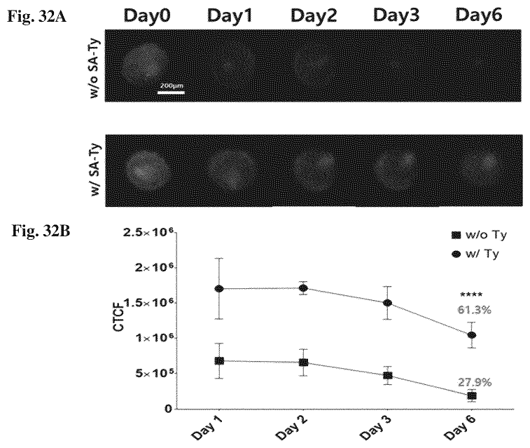

Encapsulation was performed using polymers to which fluorescent molecules were introduced, and the results were divided into an enzyme group and a non-enzyme group. After encapsulation, the changes in fluorescence intensity over the period of in vitro culture were quantified to evaluate the persistence. The results are shown in FIG. 31. Further, the quantitative analysis results are also shown in FIGS. 32A and 32B (Comparison of persistence depending on the presence or absence of enzymatic cross-linking of fluorescent polymers, (FIG. 32A) Fluorescence images, (FIG. 32B) Quantitative data of fluorescence intensity (n=3). **** P<0.0001). Fluorescence imaging of the spheroids was performed, and the fluorescence image was quantified to compare the fluorescence intensity for different incubation periods. As a result, after 6 days of incubation, the quantitative value of fluorescence intensity was 61.3% of that on the first day in the enzyme group and 27.9% in the non-enzyme group, which means that the enzyme group achieved the target value, i.e., 50%, with more than 219% of encapsulation persistence compared to the non-enzyme group, confirming the enhancement of thin film persistence by enzymatic cross-linking.

### Experimental Example 13: Anti-coagulation

It is based on the blood-mediated immune response that can occur in a liver engraftment model using the Edmonton protocol during transplantation of encapsulated pancreatic islet cells. To reduce blood clotting (thrombosis) and pancreatic islet cell loss caused by the transplant material, encapsulation of modified hydrogel capable of preventing blood clotting was applied.

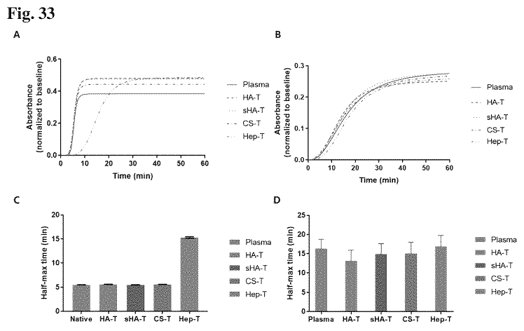

Blood was centrifuged to isolate plasma, and 0.3 wt % sulfated polymers or encapsulated PMMA beads (poly methyl methacrylate beads) were added to the isolated plasma and treated with calcium chloride to cause a coagulation. The change in absorbance at 405 nm was measured. 0.5 times the saturation value corresponds to the half-max time. The delay of plasma coagulation of each polymer was quantitatively compared. The results are shown in FIG. 33. The experiments were conducted with the non-encapsulated group and the hyaluronic acid (non-sulfated polymer) group as controls. In particular, the heparin group was found to have an excellent coagulation delay effect.

### Clotting in the Blood

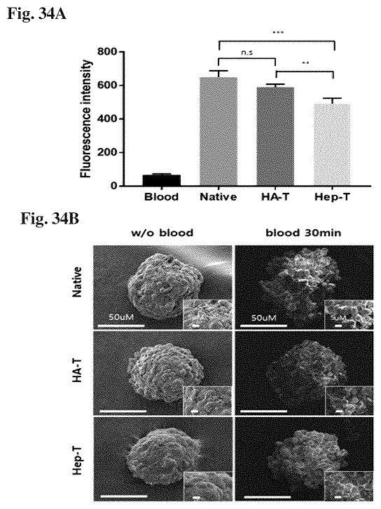

Cell spheroids encapsulated with modified hydrogels were cultured in blood and the adsorption of blood substances such as red blood cell count and thrombin activity were analyzed. Thrombin, a clotting factor in blood, is at prothrombin state and activated to thrombin by the coagulation activation pathway, inducing blood clotting. The degree of blood clotting prevention may be determined by the degree of thrombin activity increase. Spheroids encapsulated with each sulfated polymer were incubated in blood for 30 min, and then plasma was separated to analyze thrombin activity by ELISA. Lower thrombin activity was observed in the heparin-encapsulated group than in the non-encapsulated group and the hyaluronic acid-encapsulated group (FIG. 34A).

Additionally, MIN6 spheroids cultured in blood were fixed and examined by SEM to determine changes in surface and morphology, as well as adsorption of red blood cells and other substances in blood. The results are shown in FIG. 34A, thrombin activity (n=3), and 34B, SEM of spheroids. ** P<0.01). In the case of non-encapsulated spheroids, it was observed that the spheroid shape was deformed as red blood cells adhered, and it was confirmed that hyaluronic acid-encapsulated spheroids maintained the spheroid shape but had many red blood cells clumped together. In contrast, it was confirmed that heparin-encapsulated spheroids had well-maintained single cells, less red blood cell clumping and well-maintained morphology due to less blood coagulation-mediated reaction due to the heparin coating on the surface (FIG. 34B).

### Platelet Adhesion Prevention

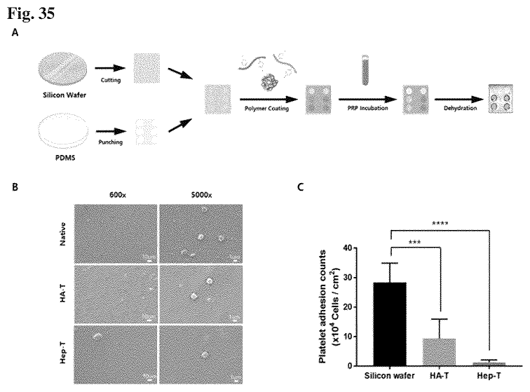

Platelets are involved in blood clotting by activating prothrombin into thrombin. When platelets aggregate on the surface of the pancreatic islet, a blood clotting reaction occurs, which reduces the survival rate. Therefore, it is necessary to reduce the degree of platelet adhesion through encapsulation modification. Thus, polymers were coated on a silicon wafer under conventional encapsulation conditions, and polymers were layered on a silicon wafer coated with platelet rich plasma (PRP) under the same conditions as the encapsulation process. Platelet rich plasma was incubated for one hour to analyze the degree of platelet surface adhesion. The results are shown in FIG. 35 ((A) SEM imaging, (B) Measurement of platelet adhesion (n=5). **** P<0.0001). The degree of platelet adhesion on each silicon wafer plate was counted from the SEM image, and it was confirmed that significantly less platelets were adhered on the heparin-coated wafer plate than on the control group.

### Experimental Example 14: Optimization of Encapsulation Process

14-1: Selection and analysis of negatively charged polymers according to sulfation degree/Optimization of the synthesis method for substituting monophenol groups in sulfated polymers and determination of cross-linking ability (qualitative analysis)

Chondroitin sulfate (CS), sulfated hyaluronic acid (sHA), and heparin (Hep) were selected as the polymer having a sulfate group (SO₃²⁻), and tyramine groups were substituted for carboxyl groups of each polymer using the EDC/NHS synthesis. Hyaluronic acid was used as a control polymer with a sulfation degree of 0.

In a previous study, it was shown that the substitution rate of monophenol groups in hyaluronic acid was 17.5%, and it was confirmed that it could be increased to 22.8% when the ratio of tyramine was increased. For the other types of sulfated polymers, enzymatic cross-linking ability was secured through sufficient monophenol group substitution. The zeta potential was measured by substituting monophenol groups for each of the selected negatively charged sulfated polymers. As a result, it was confirmed that the higher the sulfation degree, the lower the zeta potential. The results of related H-NMR analysis results and zeta potential (n=3) are shown in FIG. 29.

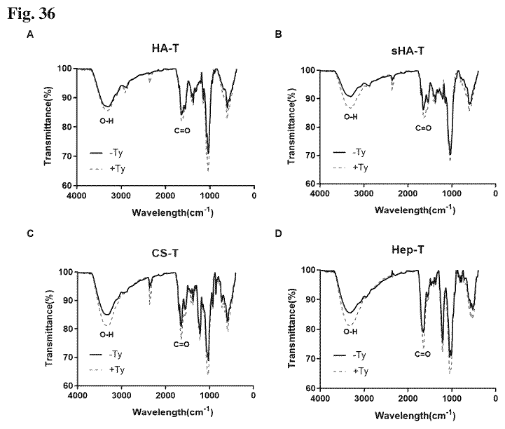

To confirm the cross-linking ability of the synthesized sulfated polymers, each was cross-linked using enzymes for 30 min, then freeze-dried and analyzed using FT-IR. It was confirmed that the C═O double bond was increased from a monophenol group to a quinone group during crosslinking (FIG. 36).

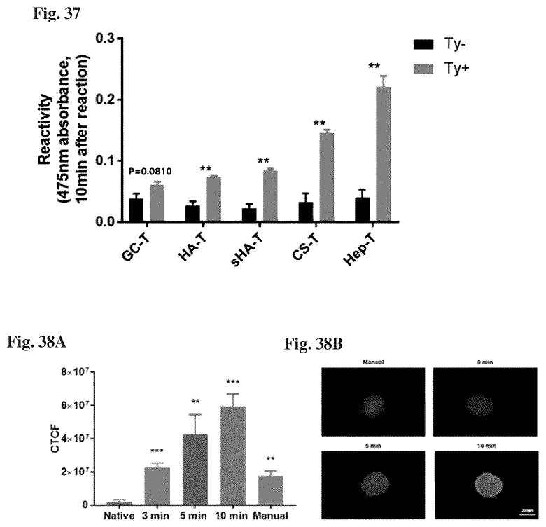

The substrate reaction rate was calculated by measuring the absorbance at 1,475 nm 10 min after enzymatic cross-linking of 0.1 wt % of sulfated polymer. It was confirmed that all polymers including glycol chitosan showed increased absorbance due to crosslinking, and the introduction of monophenol groups was cross-validated (FIG. 37, n=3, ** P<0.01).

### Experimental Example 15: Optimization of Encapsulation Time (Quantitative Analysis)

Pancreatic islet cells exist in the form of spheroids with a size of 50 μm or more, so they are less likely to be lost even when directly exposed to a solution compared to single cells. Accordingly, they were encapsulated using a cell strainer with 40 μm pore size, which allows relatively free inflow and outflow of the solution. The inflow time of the encapsulation solution was shortened by changing the slow diffusion rate due to the small pore size of the Transwell.

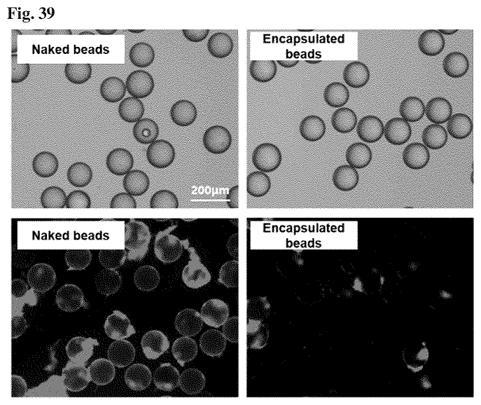

Encapsulation was performed with polymers having fluorescent molecules, and the fluorescence intensity was quantified according to the encapsulation time and compared with the conventional encapsulation using a Transwell pore size of 3 μm. Immersion in the encapsulation solution for 5 min per layering showed a higher fluorescence intensity than the conventional Transwell method. The encapsulation time including the washing process was 39 min in total, which was within the target of 45 min (FIG. 38A, n=4, and FIG. 38B, ** P<0.01, *** P<0.001).

### Experimental Example 16: Collagen Adsorption

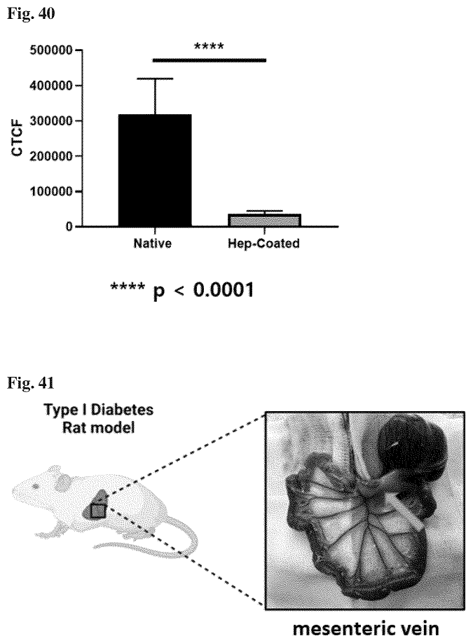

PMMA (poly methyl methacrylate) beads encapsulated with the modified anticoagulating hydrogel for cell encapsulation were incubated in 50 μg/ml of Collagen-FITC for 2 hours. Heparin as a sulfated polymer was used for encapsulation of PMMA. The non-encapsulation group was set as a control group, and the collagen adsorbed on the beads was imaged by fluorescence imaging, as shown in FIG. 39. In addition, the fluorescence intensity was quantified and presented as a graph in FIG. 40. Based on the image results and fluorescence intensity quantitative data, it was confirmed that the collagen adsorption rate on the surface of the encapsulated beads according to the present invention was significantly reduced compared to the control group. These results indicate that the introduction of modified encapsulation prevents fibrosis around transplanted cells by preventing extracellular matrix adsorption to the surface.

### Experimental Example 17: Allogeneic Pancreatic Islet Transplantation Model

Type 1 diabetes was induced in rats by intraperitoneal administration of 150 mg/kg of Alloxan. Three days later, the rats were opened after inhalation anesthesia and heparin-encapsulated pancreatic cells were transplanted into the mesenteric vein through a catheter. Blood glucose levels were monitored. A non-encapsulated pancreatic islet transplantation model and an encapsulated pancreatic islet transplantation model without sulfated polymer were used as controls for INS1 spheroids (rat β-cell line). In the non-encapsulated pancreatic islet transplantation model, 5.2 million cells of INS1 cells were transplanted, and in the encapsulated pancreatic islet transplantation model without sulfated polymer, 7.2 million cells were transplanted.

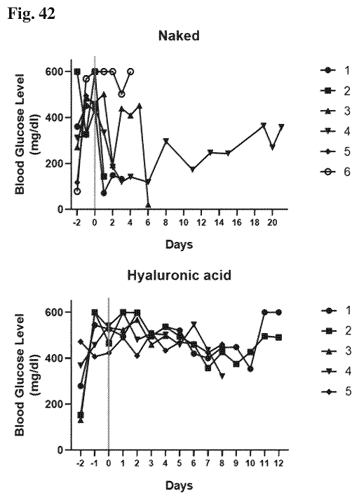

A photograph of the mesenteric vein where pancreatic islet cells were engrafted is shown in FIG. 41, and a graph of blood glucose level in the control group is shown in FIG. 42. In the control model, the experiment was terminated early due to maintenance of hyperglycemia or death from hypoglycemia.

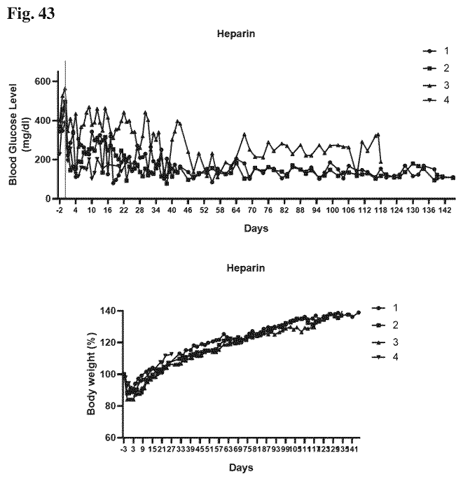

FIG. 43 is a graph showing changes in blood glucose level and body weight in the allogeneic pancreatic islet transplantation model in which 7.2 million INS1 cells, i.e., INS1 spheroids using the anticoagulating hydrogel for cell encapsulation modified with heparin as a sulfated polymer, were transplanted. From FIG. 43, it was confirmed that the group of encapsulated pancreatic islet cells according to the present invention can control blood glucose levels for a long period of 140 days or more.

Therefore, it can be seen that the heparin-modified encapsulation composition is effective for the application of the transplantation method in which pancreatic islet cells are injected into the portal vein and engrafted in the liver according to the Edmonton protocol.

As can be seen from the above results, a nanometer-thick hydrogel nanofilm can be formed on the outside of the cells without toxicity to the cells, which leads to improve stability by forming a covalent bond between the polymers constituting the nanofilm. In addition, since an appropriate thickness can be easily formed to the hydrogel nanofilm, the exchange of substances between the cells and the external environment is facilitated, thereby protecting the cells to maintain their function. In addition, by the encapsulation technology, it is possible to block the immune response that occurs when foreign cells are transplanted into the body, and by modifying the outermost layer using sulfated polymers, it is possible to provide a reduction in a blood-mediated inflammatory reaction, especially platelet adhesion that occurs upon blood contact, and anticoagulant effect. Therefore, with the encapsulation technique of the present invention, cells to be transplanted can be introduced into the body with a high survival rate, especially when injected through blood vessels.

As described above, by using the encapsulation of hydrogel whose outermost layer is modified using the sulfated polymer of the present invention, immune responses and blood-mediated inflammatory reactions occurring during cell transplantation can be effectively controlled compared to the conventional microcapsule or device method. In addition to pancreatic islet cells, it can also be applied to other endocrine cells. In addition to sulfated polymers, functional polymers into which monophenol groups are introduced can be used to control other immune responses (e.g., fibrosis) in the body. Furthermore, while conventional encapsulation requires encapsulation devices worth tens of millions to hundreds of millions of KRW, the encapsulation process using the modified hydrogel of the present invention can be performed at the laboratory level or in an operating room, thereby reducing costs and enabling the introduction of the technology without much expertise.

The above descriptions are merely illustrative of the technical idea of the present invention, and various modifications and variations are possible for those of ordinary skill in the art to which the present invention pertains without departing from the essential characteristics of the present invention. Also, the Examples described in the present invention are not intended to limit the technical spirit of the present invention, but to illustrate the invention, and thus the scope of the technical spirit of the present invention is not limited by these Examples. The protection scope of the present invention should be construed by the following claims, and all technical ideas within the scope equivalent thereto should be understood as being included in the scope of the present invention.

What is claimed is:

1. A modified anticoagulating hydrogel for cell encapsulation, comprising:
a chitosan layer comprising a functionalized chitosan;
a hyaluronic acid layer comprising a functionalized hyaluronic acid; and
an outermost layer comprising a functionalized sulfated polymer,
wherein the chitosan layer and the hyaluronic acid layer are alternately layered,
the chitosan layer and the hyaluronic acid layer form aryloxy cross-linking with each other by an enzymatic reaction of tyrosinase, and
the sulfated polymer comprises at least one of chondroitin sulfate, sulfated hyaluronic acid, and heparin.

2. The modified anticoagulating hydrogel for cell encapsulation according to claim 1, wherein the thickness of the hydrogel is 100 nm to 200 nm.

3. The modified anticoagulating hydrogel for cell encapsulation according to claim 1, wherein the chitosan, the hyaluronic acid and the sulfated polymer is each independently functionalized with phenol, catechol or quinone.

4. The modified anticoagulating hydrogel for cell encapsulation according to claim 1, wherein a monophenol group is introduced into each of the chitosan, hyaluronic acid and sulfated polymer.

5. The modified anticoagulating hydrogel for cell encapsulation according to claim 1, wherein the hydrogel for cell encapsulation comprises 1 to 5 chitosan layers and 1 to 5 hyaluronic acid layers.

6. The modified anticoagulating hydrogel for cell encapsulation according to claim 1, wherein the hydrogel is for transplantation of isolated B-cells.

7. A method for producing the modified anticoagulating hydrogel for cell encapsulation of claim 1, comprising the steps of:
(i) adding a solution containing a functionalized chitosan and a solution containing a functionalized hyaluronic acid to form layers having a chitosan layer and a hyaluronic acid layer alternately layered, wherein the chitosan layer and the hyaluronic acid layer form aryloxy cross-linking with each other by using tyrosinase enzyme; and
(ii) adding a solution containing a functionalized sulfated polymer onto the layers obtained from step (i) to form an outermost layer, wherein the outermost layer forms aryloxy cross-linking by using tyrosinase enzyme,
wherein the sulfated polymer comprises at least one of chondroitin sulfate, sulfated hyaluronic acid, and heparin.

8. The method for producing the modified anticoagulating hydrogel for cell encapsulation according to claim 7, wherein the solution containing a functionalized chitosan has a concentration of 0.01% (w/v) to 5% (w/v), the solution containing a functionalized hyaluronic acid has a concentration of 0.01% (w/v) to 5% (w/v), and the solution containing a functionalized sulfated polymer has a concentration of 0.01% (w/v) to 5% (w/v).

9. The method for producing the modified anticoagulating hydrogel for cell encapsulation according to claim 7, wherein the method is for transplantation of isolated cells.

10. The method for producing the modified anticoagulating hydrogel for cell encapsulation according to claim 7, wherein the tyrosinase enzyme includes Streptomyces avermitilis-derived tyrosinase.

11. A cell therapy composition for isolated cell transplantation, comprising the modified anticoagulating hydrogel for cell encapsulation of claim 1 and isolated cell.

12. An isolated cell encapsulated with the modified anticoagulating hydrogel for cell encapsulation of claim 1.
